(19) United States
(12) Patent Application Publication
(10) Pub. No.: US 2014/0272925 A1
(43) Pub. Date: Sep. 18, 2014

(54) Plasma Separation Apparatus, Method and System

(71) Applicant: CHROMOLOGIC LLC, Pasadena, CA (US)

(72) Inventors: Naresh Menon, Pasadena, CA (US); Gregory Bearman, Pasadena, CA (US); Cheryl Tan, Fresno, CA (US); Julian Down, Cambridge, MA (US)

(73) Assignee: ChromoLogic LLC, Pasadena, CA (US)

(21) Appl. No.: 13/830,065

(22) Filed: Mar. 14, 2013

(51) Int. Cl. B01L 3/00; G01N 33/49

(52) U.S. Cl. CPC B01L 3/5021; G01N 33/491
USPC 435/2; 435/307.1

(57) ABSTRACT

A blood fractionating apparatus that enables aseptic fractionation and storing of fractionated blood components, comprises a centrifuge tube including three chambers separated by constricted sealable passages, an open operative upper end chamber, a closed operative lower end chamber and a middle chamber defined between the upper end chamber and the lower end chamber and connected by means of the sealable passages. A stopper with a flapper valve is provided at the opening of the upper end chamber to control flow of fluid to and from the chambers. This apparatus enables separating the chambers to obtain a hermetically sealed operative lower chamber containing the corpuscle component and a hermetically sealed middle chamber containing the plasma component that can be stored at ambient temperature.

This application is a non-provisional patent application claiming priority to U.S. provisional patent application No. 61/685,320 filed on Mar. 14, 2012, entitled “PLASMA SEPARATION SYSTEM” incorporated by reference herein.

This invention was made under a contract with an agency of the United States Government, NASA Phase II Contract No. NNX11CB42C.

### FIELD OF THE DISCLOSURE

The present invention relates to an apparatus and method for fractionating blood and storing fractionated blood components.

### BACKGROUND

Blood comprises a plasma component and a blood corpuscle component. The blood corpuscle component comprises erythrocytes (i.e., red blood cells), leucocytes (i.e., white blood cells), and blood platelets. Blood plasma makes up about 60% of total blood volume. It is mostly composed of water (90% by volume), and contains dissolved proteins, glucose, clotting factors, mineral ions, hormones and carbon dioxide. Many diagnostic tests in a conventional clinical laboratory are performed on blood plasma because changes in composition of plasma often reflect the current status of pathological processes throughout the body.

Plasma is usually obtained from whole blood by centrifugation method with electrically-powered, bench-top centrifuges at speeds that generate approximately 1000 g for 15 minutes. This method sediments blood cells, which interfere with assays as they scatter light, aggregate and lyse. Further, it is challenging to separate the red and white blood cells without rupturing their cell wall, which releases intracellular proteases, which further can destroy proteins present in the plasma. According to the above method, the plasma thus separated can be dispensed using a pipette or by decantation. Great care must be taken during manual handling of these separated blood components to avoid microbial contamination or undesirable re-mixing of the corpuscle component in the plasma.

Most prior art methods for the separation of plasma are limited with respect to speed, yield efficiency and they also do not permit room temperature storage. There exists a need for development of a rapid and efficient method for plasma separation. Accordingly, there is a need for a quick, inexpensive and simple apparatus that could ameliorate the problems known in the art.

### DEFINITIONS

The expression “fluid” used in the specification refers to but is not limited to blood samples and gases including air.

The expression “blood sample” used in the specification refers to but is not limited to biological samples with components that can be sedimented, blood in its original composition, partially fractionated blood and a blood sample comprising at least one additive which includes but is not limited to anti-coagulants, sedimentation aids and anti-adhesive agents.

The expression “sedimentation aids” used in the specification refers to but is not limited to additives including microspheres, assay beads, reactive beads, magnetic beads, dye coated beads and the like, that serve to capture the specific blood component, under consideration, from the blood sample.

The expression “corpuscle” used in the specification refers to but is not limited to erythrocytes, leucocytes, blood platelets and the like.

These definitions are in addition to those expressed in the art.

### SUMMARY

The present invention provides for fractionating blood and storing fractionated blood components; blood fractionating apparatus fractionates and stores fractionated blood components, said apparatus comprising: a centrifuge tube defined by at least three chambers separated by constricted passages that are sealable: an open operative upper end chamber, a closed operative lower end chamber and a middle chamber defined between the upper end chamber and the lower end chamber and connected via the sealable passages; and a stopper with a flapper valve provided at the opening of the upper end chamber, the flapper valve adapted to control flow of fluid to and from the chambers.

In accordance with the present invention, the blood fractionating apparatus as described herein above further comprises sealing means to hermetically seal the sealable passages and separate the hermetically sealed chambers from each other. Said sealing means may further comprise a crimping means for aiding in the sealing of the sealable passages, or constricting flow therethrough.

Typically, the tube is made of a polymeric material selected from the group consisting of polyurethane, thermo-plastics and elastomers.

Preferably, the tube is pre-coated with at least one of anti-coagulant and anti-adhesive agents. The anti-coagulant is meant to prevent clotting of the blood sample and the anti-adhesive coating facilitates minimizing of protein adhesion to the inner surface of the tube.

In accordance with the present invention, there is provided a process for fractionating a blood sample into a plasma component and a corpuscle component and storing the fractionated components; the process comprising the following steps:
providing a blood fractionating apparatus comprising: a centrifuge tube defined by at least three chambers separated by constricted sealable passages: an open operative upper end chamber, a closed operative lower end chamber and a middle chamber defined between the upper end chamber and the lower end chamber and connected via the sealable passages; and a stopper with a flapper valve provided at the opening of the upper end chamber, the flapper valve adapted to control flow of fluid to and from the chambers;
evacuating air from the tube to maintain a predetermined pressure inside the tube;
injecting the blood sample into the tube via the stopper;
filling the tube with the blood sample up to a predetermined level;
centrifuging the tube at a predetermined speed for a predetermined time period to drive the corpuscle component into the lower end chamber leaving the plasma component in the middle chamber; and
sealing the sealable passages to separate the chambers to obtain a hermetically sealed operative lower chamber containing the corpuscle component and a hermitically sealed middle chamber containing the plasma component.

Preferably, the step of providing a blood fractionating apparatus further includes the step of coating at least one of anti-coagulant and anti-adhesive agents to an inner surface of the tube. The anti-coagulant is meant to prevent clotting of the blood sample and the anti-adhesive coating facilitates minimizing of protein adhesion to the inner surface of the tube.

Typically, in accordance with the present invention, the step of evacuating air, described herein above, is performed by purging the tube with Nitrogen gas.

Typically, in accordance with the present invention, the step of evacuating air is performed to maintain pressure within the tube at 2 atm.

Optionally, the step of injecting the blood sample further comprises the step of injecting at least one additive selected from the group consisting of anti-coagulants, sedimentation aids and anti-adhesive agents.

Typically, in accordance with the present invention, the step of centrifuging is performed at a speed ranging between 1500 and 2500 rpm and for a time period ranging between 10 and 20 min.

In accordance with the present invention, the step of sealing, described herein above, comprises at least one of mechanically crimping and sealing, thermal cutting and sealing, thermally melding and sealing, ultrasound welding, UV sealing, chemically sealing and epoxy based sealing.

It is an object of the present invention to ameliorate one or more problems of the prior art or to at least provide a useful alternative.

It is another object of the present invention is to provide an apparatus and method for rapid blood fractionation.

It is still another object of the present invention is to provide a cost effective apparatus and method for blood fractionation.

It is further object of the present invention is to provide an efficient apparatus and method for blood fractionation.

An additional object of the present invention is to provide an apparatus and method for blood fractionation that aseptically separates the plasma component and the corpuscle component that can be stored at ambient temperature.

A further object of the present invention is to provide a reliable apparatus and method for blood fractionation.

A still further object of the present invention is to provide a simple apparatus and method for blood fractionation.

Other objects and advantages of the present invention will be more apparent from the following description when read in conjunction with the accompanying figures, which are not intended to limit the scope of the present invention.

### BRIEF DESCRIPTION OF THE ACCOMPANYING DRAWINGS

The blood fractionating apparatus of the present invention will now be explained in relation to the accompanying drawings, in which:

FIG. 1 illustrates a blood fractionating apparatus in accordance with an embodiment of the present invention;

FIGS. 2A, 2B and 2C illustrate a sectional side view of a stopper with an open flapper valve, a sectional side view of a stopper with a closed flapper valve and a perspective top view of a stopper with a flapper valve provided at an operative upper end chamber of the apparatus of FIG. 1;

FIG. 3 illustrates three configurations of the apparatus of FIG. 1 corresponding to different blood sample volumes;

FIGS. 4A, 4B and 4C illustrate three of the stages in the process for obtaining a hermetically sealed chamber including the fractionated plasma component from the blood sample;

FIG. 5 illustrates an evacuated apparatus of FIG. 1 at varying time intervals;

FIG. 6 illustrates comparative analyses of amount of total protein, albumin, albumin/globulin ratio and globin in g/dL using 4 different methods; and

FIG. 7 illustrates comparative analyses of amount of alkaline phosphatase, aspartase aminotransferase, alanine aminotransferase, creatinine kinase and gamma glutamyl transferase in g/dL using 4 different methods.

### DETAILED DESCRIPTION

A preferred embodiment will now be described in detail with reference to the accompanying drawings. The preferred embodiment does not limit the scope and ambit of the invention. The description provided is purely by way of example and illustration.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The following description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The present invention envisages a blood fractionating apparatus to fractionate a blood sample into a plasma component and a corpuscle component. The apparatus of the present invention also facilitates aseptically storing the fractionated blood components in hermetically sealed chambers.

Blood plasma makes up about 60% of total blood volume. It primarily constitutes water to the extent of about 90% by volume and about 10% dissolved proteins, glucose, clotting factors, mineral ions, hormones and carbon dioxide. The blood fractionating apparatus known in the art are plagued by problems associated with maintaining the sterility of the separated plasma and corpuscle components.

The blood fractionating apparatus of the present invention is illustrated in FIG. 1 and for ease of explanation, the main parts of the apparatus are generally referenced by numerals as indicated herein below:
a blood fractionating apparatus 100;
a centrifuge tube 10;
a stopper with flapper valve 12;
constricted sealable passages 14;
an operative upper end chamber 16;
a middle chamber 18;
an operative lower end chamber 20; and
a fill line 22 for the blood sample.

The blood fractionating apparatus 100 of the present invention comprises a centrifuge tube 10. The tube 10 is typically made of a polymeric material, preferably, plastic, and is provided with a plurality of constricted sealable passages 14 in a spaced apart configuration. Alternatively, the polymeric material includes at least one of polyurethane, thermo-plastics, and elastomers. The stopper with a flapper valve 12 is sealingly provided at the opening of the operative upper end chamber 16. The stopper serves to enable injection of a blood sample into the tube 10. The flapper valve serves to maintain positive pressure inside the tube 10. Air in the tube 10 is evacuated typically by purging the tube 10 with an inert gas, for instance, Nitrogen. The pressure inside the tube is maintained at a predetermined value below atmospheric pressure to ensure positive pressure and create suction when a blood sample is injected into the tube 10. Typically, the pressure within the tube is maintained at 2 atmosphere (atm).

When a blood sample, is centrifuged, the result is that the corpuscle component typically settles to the bottom with the plasma component disposed above the corpuscle component. The apparatus 100 of the present invention is based on this principle. It is provided with the constricted sealable passages 14 in such a way that at least three chambers are defined within the tube 10. The blood sample is injected into the tube 10 via the stopper having a self-sealing injection site (not shown). The suction within the tube 10 enables flow of the blood sample from the operative upper end chamber 16 into the operative lower end chamber 20 via the middle chamber 18 until the tube 10 is filled up to a predetermined level that is defined in the first segment 16 and indicated by the fill line 22.

The tube 10 is then centrifuged at a predetermined speed for a predetermined time period to fractionate the blood sample. The plasma component is contained in the middle chamber 18 and the corpuscle component is contained in the operative lower chamber 20.

The operative upper end chamber 16, the middle chamber 18 and operative lower chamber 20 are then hermetically sealed and separated by a sealing means 24. Typically, the sealing means is at least one of mechanical crimping and sealing, thermal cutting and sealing, thermally melding and sealing, ultrasound welding, chemical sealing, epoxy based sealing and UV sealing methods.

The step of centrifuging is typically performed at a speed ranging between 1500 and 2500 rpm and for a time period ranging between 10 and 20 min.

FIGS. 2A, 2B and 2C illustrate a sectional side view of the stopper 12 with an open flapper valve, a sectional side view of a stopper 12 with a closed flapper valve and a perspective top view of a stopper 12 with the flapper valve provided at an operative upper end chamber of the apparatus 100 described herein above. The flapper valve can be opened as illustrated in FIG. 2A to expel air only in the case of increased pressure inside the tube 10.

FIG. 3 illustrates three configurations of the apparatus 100 of FIG. 1 corresponding to different blood sample volumes. A scale is illustrated for dimensional reference only. The blood sample volume corresponds to the length of the centrifuge tube 10 of FIG. 1 and the placement of the sealable constricted passages 14 is decided based on the blood sample volume and the predicted volume of the plasma component and the corpuscle component constituting the blood sample.

FIGS. 4A, 4B and 4C illustrate three of the stages in the process for obtaining a hermetically sealed chamber including the fractionated plasma component from the blood sample. FIG. 4A illustrates the tube 10 filled with a blood sample. When the tube 10 is subjected to centrifugation at a predetermined speed and time period, the blood sample contained within the tube 10 is fractionated and it results in the separation of the plasma component and the corpuscle component as illustrated in FIG. 4B. The constricted sealable passages 14 are then sealed to obtain hermetically sealed chambers containing the fractionated blood components. FIG. 4C specifically illustrates a hermetically sealed middle chamber 18 containing the plasma component.

In accordance with the present invention, the tube 10 is provided with at least one of an anti-coagulant and an anti-adhesive coating. The anti-coagulant is meant to prevent clotting of the blood sample and the anti-adhesive coating facilitates minimizing of protein adhesion to the inner surface of the tube. Optionally, an anti-coagulant, such as Heparin, may be added in liquid or dry form to the blood sample before or after injecting the blood sample into the tube 10. The anti-adhesive coating is typically epoxy-silane or any other hydrophilic coating.

FIG. 5 illustrates an evacuated apparatus of FIG. 1 at varying time intervals. FIG. 5 shows that the tube 10 when evacuated, collapses into a compact form that facilitates easy storage. FIG. 5 also illustrates that the tube 10 maintains its collapsed state even 240 hours after it was evacuated.

FIG. 6 illustrates comparative analyses of amount of total protein, albumin, albumin/globulin ratio and globin in g/dL using 4 different methods, wherein (I) represents total protein, (II) represents albumin, (III) represents albumin/globulin ratio and (IV) represents globin respectively.

FIG. 7 illustrates comparative analyses of amount of alkaline phosphatase, aspartase aminotransferase, alanine aminotransferase, creatinine kinase and gamma glutamyl transferase in g/dL using 4 different methods, wherein (I) represents alkaline phosphatase, (II) represents aspartase aminotransferase, (III) represents alanine aminotransferase, (IV) represents creatinine kinase and (V) represents gamma glutamyl transferase respectively.

(A) represents a method known in the art, wherein the fractionated plasma component is pipetted out and stored at −80 deg C. (B) represents a method in accordance with the present invention wherein the fractionated plasma component is hermetically sealed and separated and stored at ambient temperature. (C) represents a method wherein the fractionated plasma component is pipetted and transferred to the apparatus of the present invention for hermetically sealing and storing at ambient temperature. Method (C) serves as a positive control. (D) represents a method wherein the fractionated plasma component is contaminated with 1% of lysed blood cells. Method (D) serves as a negative control. The results show that after 62 days, the method (B) in accordance with the present invention is superior to the Method (A) known in the art, with regards to preserving proteins in whole blood.

The blood fractionating apparatus of the present invention does not need any human handling and thus eliminates contamination of the fractionated components and also eliminates possibility of oxygen or other contaminants being introduced into the tube to contaminate the contents, for example, through the growth of bacteria. This further permits storing of the aseptic fractionated components at ambient temperature. The blood fractionating apparatus of the present invention finds application in low resource medical care, natural disaster and casualty, analytical clinical labs, population studies and the like.

Table 1 below summarizes the measured difference between samples consisting of whole bovine blood stored with the present invention (fluid preservation system—FPS) stored at room temperature and the conventional method (gold standard) for separating plasma by centrifugation and freezing it at −80 C (negative 80 degrees Celsius).

TABLE 1

[chemical structure]

Samples stored with invented containers and those stored with the gold standard method were analyzed by a “Chem 21” analysis panel. This standard clinical chemistry panel measures various components in whole blood and is widely used for broad clinical diagnostic assessment. The panel includes measurement of protein concentration, enzymatic activity, salt content and gasses. The data shows that the invented containers are consistent with the gold standards for up to 8 days for all analytes in whole blood except for liver enzymatic activity (AST, ALT, Creatinine Kinase) and total glucose. The present embodiment may not perform as well for the preservation of enzymes (shown in grey in Table 1). In that event, enzyme-preserving reagents can be included with the invented containers to preserve the enzymatic activity as an additional embodiment.

Table 2 below shows the outcome of preserving proteins in whole (bovine) blood for 62 days by 4 different methods.

Method 1 (−80 STD) consists of extracting plasma by centrifuging the starting whole blood and manually pipetting plasma into a capsule followed by freezing it at −80 C (negative 80 degrees Celsius). Method 2 (RT STD FPS) consists of the current embodiment of the invention where the plasma was separated, sealed and stored at room temperature. Method 3 (RT PST FPS*) consists of a hybrid version of methods 1 and 2. In this method, the whole blood sample was centrifuged in a standard test tube (as in Method 1), separated manually with a pipette and sealed/stored at room temperature with the a manifestation of the invention (Method 2). Method 3 serves as a positive control. In Method 4 (RT 1% LBC FPS***), a sample prepared by method 1 was mixed in with 1% of lysed blood cells, which serves as a contaminant. Method 4 serves as a negative control. The results show that after 62 days, the invention (Method 2; RT STD FPS) is superior to the gold standard (Method 1; −80 STD) when it comes to preserving proteins in whole blood.

### TECHNICAL ADVANCEMENTS AND ECONOMIC SIGNIFICANCE

The technical advancements offered by the blood fractionating apparatus of the present invention include the realization of:

an apparatus and method for rapid blood fractionation;

a cost effective apparatus and method for blood fractionation;

an efficient apparatus and method for blood fractionation;

an apparatus and method for blood fractionation that aseptically separates the plasma component and the corpuscle component that can be stored at ambient temperature thereby eliminating the need for refrigeration/refrigerants during shipping and storage;

a reliable apparatus and method for blood fractionation; and

a simple apparatus and method for blood fractionation.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results.

The numerical values mentioned for the various physical parameters, dimensions or quantities are only approximations and it is envisaged that the values higher/lower than the numerical values assigned to the parameters, dimensions or quantities fall within the scope of the invention, unless there is a statement in the specification specific to the contrary.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

1. A blood fractionating apparatus for fractionating and storing fractionated blood components, said apparatus comprising: a centrifuge tube defined by at least three chambers separated by constricted sealable passages: an open operative upper end chamber, a closed operative lower end chamber and a middle chamber defined between said upper end chamber and said lower end chamber and connected via said sealable passages; and a stopper with a flapper valve provided at the opening of said upper end chamber, said flapper valve adapted to control flow of fluid to and from said chambers.

2. A blood fractionating apparatus for fractionating and storing fractionated blood components, said apparatus comprising: a centrifuge tube defined by at least three chambers separated by constricted sealable passages: an open operative upper end chamber, a closed operative lower end chamber and a middle chamber defined between said upper end chamber and said lower end chamber and connected via said sealable passages; and a means for closing said opening of said upper end chamber, and means for controlling flow of fluid to and from said chambers.

3. The blood fractionating apparatus as claimed in claim 1 further comprising sealing means to crimp and hermetically seal said sealable passages and separate the hermetically sealed chambers from each other.

4. The blood fractionating apparatus as claimed in claim 1, wherein said tube is made of a polymeric material selected from the group consisting of polyurethane, thermo-plastics, and elastomers.

5. The blood fractionating apparatus as claimed in claim 1, wherein said tube is pre-coated with at least one of anti-coagulant and anti-adhesive agents.

6. A process for fractionating a blood sample into a plasma component and a corpuscle component and storing the fractionated components; said process comprising the following steps:
providing a blood fractionating apparatus comprising: a centrifuge tube defined by at least three chambers separated by constricted sealable passages: an open operative upper end chamber, a closed operative lower end chamber and a middle chamber defined between said upper end chamber and said lower end chamber and connected via said sealable passages; and a stopper with a flapper valve provided at the opening of said upper end chamber, said flapper valve adapted to control flow of fluid to and from said chambers;
evacuating air from said tube to maintain a predetermined pressure inside said tube;
injecting the blood sample into said tube via said stopper;
filling said tube with the blood sample up to a predetermined level;
centrifuging said tube at a predetermined speed for a predetermined time period to drive the corpuscle component into said lower end chamber leaving the plasma component in said middle chamber; and
sealing said sealable passages to separate said chambers to obtain a hermetically sealed operative lower chamber containing the corpuscle component and a hermitically sealed middle chamber containing the plasma component.

7. The process for fractionating the blood sample as claimed in claim 6, wherein the step of providing a blood fractionating apparatus further includes the step of coating at least one of anti-coagulant and anti-adhesive agents to an inner surface of said tube.

8. The process for fractionating the blood sample as claimed in claim 6, wherein the step of evacuating air is performed by purging said tube with Nitrogen gas.

9. The process for fractionating the blood sample as claimed in claim 6, wherein the step of evacuating air is performed to maintain pressure within said tube at 2 atm.

10. The process for fractionating the blood sample as claimed in claim 6, wherein the step of injecting the blood sample further comprises the step of injecting at least one additive selected from the group consisting of anti-coagulants, sedimentation aids and anti-adhesive agents.

11. The process for fractionating the blood sample as claimed in claim 6, wherein the step of centrifuging is performed at a speed ranging between 1500 and 2500 rpm.

12. The process for fractionating the blood sample as claimed in claim 6, wherein the step of centrifuging is performed for a time period ranging between 10 and 20 min.

13. The process for fractionating the blood sample as claimed in claim 6, wherein the step of sealing comprises at least one of mechanically crimping and sealing, thermal cutting and sealing, thermal melding and sealing, ultrasound welding, chemical sealing, epoxy based sealing and UV sealing methods.
